(19) United States
(12) Patent Application Publication
(10) Pub. No.: US 2004/0253633 A1
(43) Pub. Date: Dec. 16, 2004

(54) Raman techniques for detecting molecular interactions in molecularly imprinted polymers

(76) Inventors: Ala Hayall Reson Al-Obaidi, Alness (GB); Daniel McStay, Alness (GB); Peter John Quinn, The Strand London (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/333,531

(22) PCT Filed: Jun. 20, 2001
(86) PCT No.: PCT/GB01/03278

(30) Foreign Application Priority Data
Jul. 21, 2000 (GB) 0018002.6

(51) Int. Cl. G01N 33/53

(52) U.S. Cl. 435/7.1

(57) ABSTRACT

The present invention describes a novel optical technique for detecting and quantifying the binding of molecules to selective binding sites on synthetically formed molecular imprinted polymers using Raman Spectroscopy. Such a technique has immense application to the pharmaceutical and environmental industries.

This invention relates to a novel optical technique for detecting and quantifying the binding of molecules to selective binding sites on synthetic polymers. Such a technique has immense application to molecular imprinting, monitoring of drug transfer through membranes and as an alternative to radioimmunoassays.

Molecular imprinting is a technique for the preparation of polymers with a predetermined specificity for a given molecule. Essentially, molecularly imprinted polymers (MIP's) may be prepared either covalently or non-covalently. In the covalent approach, which relies on reversible covalent bonds, the imprint or template molecule is covalently attached to a polymerisable molecule. After copolymerisation with a cross linking monomer the template is chemically cleaved or otherwise removed from the highly cross-linked polymer. In the non-covalent approach, the imprint molecules are mixed with a functional monomer which is capable of interacting non-covalently with them. The functional monomers are then copolymerised with a high concentration of cross linker to yield a highly cross linked, rigid polymer. The imprint molecules are then washed from the polymer to leave cavities that are complementary to the imprint species in both shape and functionality. Methacrylic acid (MAA) has been most widely used as a functional monomer and is capable of ionic interactions with primary amines and hydrogen bonding with carboxylic acids, carbamates, amides and alcohols. Other functional monomers used include vinyl pyridine and ethylene glycol dimethacrylate (EGDMA).

The importance of MIP's has been reviewed in Kempe, M. and Mosbach, K. (1995) J. of Chromatography A 694, 3-13, Takeuchi, T. and Matsui, J. (1997) Acta Polymer 47, 471-480, Mosbach, K. and Ramstrom, O. (1996) Biotechnology 14, 163-169 and Mosbach, K. (1994) TIBS (Jan) 9-14 where it is reported that MIP's have been applied in racemic separation, enzyme and antibody mimics and sensor devices.

Despite these wide applications, there is a clear need for MIP's to be adapted to develop effective sensing systems for detecting and quantifying the interactions involving biological molecules. MIP's have been well characterised in respect of their high specificity for ligands and detection of the dynamics of binding using radioactive ligands and P2T micro-balance.

We have found that Raman spectroscopy provides a more effective optical technique for detecting and quantifying the binding of molecules to selective binding sites on synthetic MIP's, with less sensitivity to cracks which frequently occur upon the surfaces of MIP's. Similarly, Raman spectroscopy provides an effective technique for monitoring the degree of binding of templates in a MIP and a suitable system for detecting and quantifying drugs in solution.

Raman spectroscopy explores energy levels by examining the frequencies present in the light scattered by molecules. This technique involves passing a monochromatic (single frequency) incident beam consisting of a stream of photons of the same energy through a sample. Some of these photons will then collide with the molecules which will either give up some of their energy and emerge in a different direction with a lower energy and therefore a lower frequency, or these photons may collect energy from the molecules (if they are already excited), and emerge at a higher frequency. Whatever the fate of the photons, the resultant spectra give an accurate fingerprint of molecular structure at any given time.

Thus, according to the present invention we provide a novel process for detecting interactions involving a biological molecule which comprises the preparation of a polymer molecularly imprinted with a biological molecule and the use of Raman spectroscopy applied to said polymer as an optoelectronic technique for sensing said interactions. For example, the process can be employed for detecting interactions between biological molecules.

References to biological molecules will include antibodies, antigens, polynucleotides, enzymes, enzyme substrates and analogues, receptors and ligands and drugs (including potential drugs) or other pharmaceutically useful substances.

As one aspect of the present invention, we provide a novel process for detecting interactions between antibodies and antigens which comprises the preparation of a polymer molecularly imprinted with an antigen to form an artificial antibody and the use of Raman spectroscopy applied to said polymer as an optoelectronic technique for sensing interactions between antigens and the artificial antibody.

Typically, the MIP will act as an artificial antibody by incubation of functional monomers with an antigen to enable the essential features and likely binding site for a particular antigen within an antibody to be artificially synthesised. After polymerisation, the imprint molecule (antigen) may then be washed from the complex to leave cavities which are complementary to the original antigen molecule. Raman spectroscopy may then be used to detect any subsequent binding of the antigen to the artificial antibody formed by the MIP.

It will be understood that such a process will possess benefits in respect of the normal route of natural antibody preparation, eg. as the polymers are physically and chemically more resistant than natural antibodies, the polymers may be sterilised and will have a higher stability in respect of recognition properties. Additionally, the production cost will be considerably lower as the use of MIP's will eliminate the need for host animals for antibody production and will also reduce the time taken to obtain antibodies.

Preferably, the interaction between antigens and the artificial antibodies may be quantified such that an association constant (K_{A}) for the interaction may be derived.

According to a further aspect of the present invention, we provide a novel process for detecting interactions between enzymes and enzyme substrates which comprises the preparation of a polymer molecularly imprinted with a substrate to form an artificial enzyme and the use of Raman spectroscopy applied to said polymer as an optoelectronic technique for sensing interactions between substrates and the artificial enzyme.

It will be appreciated that references to substrates also include substrate analogues. Such analogues will include inhibitors and natural or non-natural substrates (eg. drug molecules).

Typically, the MIP will act as an artificial enzyme by incubation of functional monomers with a substrate to enable the essential features and likely binding site of a particular substrate within an enzyme to be artificially synthesised. After polymerisation, the imprint molecule (substrate) may then be washed from the complex to leave cavities which are complementary to the original substrate molecule. Raman spectroscopy may then be used to detect any subsequent binding of the substrate to the artificial enzyme formed by the MIP.

Preferably, the interaction between substrates and the artificial enzyme may be quantified such that an enzyme inhibition constant (K_{I}) for the interaction may be derived.

According to a further aspect of the present invention, we provide a novel process for detecting interactions between receptors and ligands which comprises the preparation of a polymer molecularly imprinted with a ligand to form an artificial receptor and the use of Raman spectroscopy applied to said polymer as an optoelectronic technique for sensing the interactions between ligands and the artificial receptor.

Typically, the MIP will act as an artificial receptor by incubation of functional monomers with a ligand to enable the essential features and likely binding site for a particular ligand within a receptor to be artificially synthesised. After polymerisation, the imprint molecule (ligand) may then be washed from the complex to leave cavities which are complementary to the original ligand molecule. This technique of preparation and detection will be beneficial when compared to natural isolation and preparation of receptors, as receptors are usually complex protein molecules resistant to normal isolation and purification procedures. Raman spectroscopy may then be used to detect any subsequent binding of the ligand to the artificial receptor formed by the MIP.

It will be appreciated that the ligands may exert an agonistic or antagonistic effect at the artificial receptor.

Preferably, the interaction between ligands and the artificial receptor may be quantified such that an association constant (K_{A}) for the interaction may be derived.

MIP's may be prepared according to known procedures, such as those described in Sellergren, B. (1997) Trends in Analytical Chemistry 16 (6), 310-320 and Mayes, A. G. and Mosbach, K. (1997) Trends in Analytical Chemistry 16 (6), 321-332.

MIP's may be prepared either covalently or non-covalently, preferably non-covalently in the presence of one or more functional polymers and a biological molecule.

It will be understood that the choice of functional monomers will depend upon the chemical groups required for artificial construction of the active site of a biological molecule. For example, methacrylic acid is a suitable functional polymer to construct an active site containing amine groups.

Suitable functional polymers will include methacrylic acid, ethylene glycol dimethacrylate or azoisobutyronitrile. Preferably the functional polymers will comprise methacrylic acid, ethylene glycol dimethacrylate and azoisobutyronitrile. Most preferably the function polymers will consist of methacrylic acid, ethylene glycol dimethacrylate and azoisobutyronitrile.

We prefer that polymerisation will occur at suitable conditions eg. low or high temperatures and in the absence of solvent. Additionally, polymerisation may occur using UV irradiation or electro-polymerisation. The biological molecule will then preferably be cleaved from the MIP, or otherwise removed, by the addition of a lower alkyl alcohol, preferably ethanol, most preferably cleavage will be assisted by mechanical agitation, ultrasound, shock waves, or any other suitable agitation means. Mechanical agitation is an especially preferred means of assisting cleavage.

Preferably, the polymer will be present as bulk polymer, a thin film, be ground up as particles or polymerised directly into spherical particles using a suitable process eg. polymerisation in emulsion.

The sensitivity of Raman spectroscopy for detecting the binding of a biological molecule to the MIP provides an optimum process for detecting interactions involving a biological molecule (eg. interactions between biological molecules), by providing a molecular fingerprint of a molecule at any given moment.

Therefore, we provide a process for detecting interactions involving a biological molecule which comprises:

(a) preparing a MIP capable of functioning as a complement to a biological molecule by incubation of functional monomers with said biological molecule;

(b) removal of the biological molecule from the MIP;

(c) comparing the Raman spectra of the MIP with the Raman spectra of a non-imprinted polymer to determine the wavelength of the peak associated with the biological molecule (“relevant peak”);

(d) introducing the MIP into an assay mixture which contains the biological molecule, incubating it therein and then obtaining a Raman spectrum of the MIP; and

(e) comparing the intensity of the relevant peak on the Raman spectra obtained from incubation of the MIP with the biological molecule in step (d) with the relevant peak obtained from the Raman spectra of the MIP alone in step (b);

wherein any change in intensity of the relevant peak will indicate existence of an interaction between the biological molecule and the complement of the biological molecule.

It will be appreciated that wherein the biological molecule and the complement of the biological molecule are enzymes and substrates, the process provides a novel technique for performing enzyme binding assays. Similarly, analogues of substrates may be used in such enzyme binding assays.

Thus, we present a process for performing enzyme binding assays wherein the biological molecule and the complement of the biological molecule are enzymes and substrates.

We also present a process for performing enzyme binding assays wherein the biological molecule and the complement of the biological molecule are enzymes and substrate analogues (eg. enzyme inhibitors).

It will also be appreciated that wherein the biological molecule and the complement of the biological molecule are receptors and ligands, the process provides a novel technique for monitoring conformational changes which may occur upon such binding, eg. monitoring the transfer of drugs through membranes.

Thus, we present a process for monitoring conformational changes wherein the biological molecule and the complement of the biological molecule are receptors and ligands.

Typically, immunoassays are used to quantify an amount of a test sample (eg. antigen) by addition of a specific reagent (eg. antibody) which will result in the formation of a bound complex. Subsequent identification of the proportion of bound reagent will indicate the quantity of the original test sample. However, this technique requires discrimination between bound and unbound complexes for quantification which can either be extremely labour intensive and time consuming or may involve the use of undesirable and hazardous radioisotopes.

Thus, we provide a novel technique for performing immunoassays wherein the biological molecule and the complement of the biological molecule are antibodies and antigens.

We also provide a process for quantifying the amount of a biological molecule present in a given environment which comprises:

(a) preparing a MIP capable of functioning as a complement to a biological molecule by incubation of functional monomers with said biological molecule;

(b) removal of the biological molecule from the MIP;

(c) comparing the Raman spectra of the MIP with the Raman spectra of a non-imprinted polymer to determine the wavelength of the peak associated with the biological molecule (“relevant peak”);

(d) measuring the intensity of the relevant peak on the Raman spectra of a range of concentrations of the polymer molecularly imprinted with the biological molecule;

(e) plotting a graph of relative peak intensity of the relevant peak against concentration of the biological molecule;

(f) introducing the MIP into an assay mixture which contains an unknown concentration of the biological molecule, incubating it therein and then obtaining a Raman spectrum of the MIP;

(g) calculating the concentration of the biological molecule by reference of the relative intensity of the relevant peak in (f to the graph constructed in (e).

It will be appreciated that when the biological molecule represents a drug or other pharmaceutically useful substance, this process will be suitable for detecting the presence of said drug or similar pharmaceutically useful substance and quantifying the amount of said drug or similar pharmaceutically useful substance.

Thus, we provide a process for quantifying the amount of a biological molecule present in a given environment wherein said biological molecule is a drug or other pharmaceutically useful substance.

It will be appreciated that the present invention is also suitable for the detection of any impurities present within a given environment (eg. detection of pesticides, insecticides or any other toxins present in water). Thus, the present invention is of immense application to the environmental industry as well as the pharmaceutical industry.

Abbreviations

EGDMA ethylene glycol dimethacrylate

MAA methacrylic acid

BOC tert butoxycarbonyl

AIBN azoisobutyronitrile

MIP molecularly imprinted polymer

bpy 2,2′ bi-pyridyl

### EXAMPLES

Raman Spectroscopy Experimental System

In all following examples the Raman spectroscopy system was used as follows: The system used a 6.5 W Ar⁺ laser (Coherent-Innova model 90) which produces a number of laser lines in the UV-visible region (351, 363, 454, 458, 465, 472, 478, 488, 496, 501, 514 and 528 nm). For the red excitation, a Ti-sapphire laser output (Spectra-Physics model 3900) pumped with the above Ar⁺ laser was used to produce a tuneable laser in the region 680-1150 nm with a maximum power of 0.8 W. The laser line of interest was filtered using a 1 nm band pass filter. Prisms and lenses were used to direct and focus the laser beam at the sample. The laser spot size at the sample is about 25 μm in diameter, which is imaged at the slit of the spectrograph (Oriel-multispec model MS125). A fixed slit width of 50 μm was used. The Raman signal was filtered before passing through spectrograph using either a Notch filter or sharp-cut coloured glass filters. The Raman spectra was detected using an air-cooled CCD camera (Oriel-Andor CCD model DU401) and then analysed using a PC computer. A schematic diagram of the Raman spectroscopy experimental system is shown in FIG. 1.

### Example 1

### Preparation of MlP's

A) Preparation of Materials

Ethylene glycol dimethacrylate (EGDMA; 98% purity) was passed through a column of basic alumina to remove inhibitors. Methacrylic acid (MM; 98% purity) was purified by distillation at reduced pressure. Azoisobutyronitrile (AIBN; 98% purity) was recrystallised from diethyl ether. All solvents used were dried and stored over 4 Å molecular sieves.

B) Polymerisations

Blocks of polymer were prepared in test tubes at room temperature (˜20° C.) under UV radiation at 366 nm. Polymerisation at low temperature is preferable because hydrogen bonding between template and monomer is favoured. Polymers imprinted with triazine and BOC-phenylalanine were formed in the absence of solvent.

C) Non Imprinted polymer

MAA (0.29 g, 0.0035 moles), EGDMA (3.36 g, 0.0169 moles) and AIBN (0.5 moles) were mixed together, purged with nitrogen for 30 minutes and placed under UV light for 16 hours to yield a transparent colourless block of polymer.

D) Triazine MIP MAA (0.29 g, 0.0035 moles), EGDMA (3.36 g, 0.0169 moles) and triazine (0.05 g, 0.0006 moles) were mixed with AIBN (0.5 moles). After purging with nitrogen for 30 minutes the monomer mixture was placed under UV light for 16 hours to yield a transparent, colourless block of polymer.

E) BOC-phenylalanine MIP

MAA (0.29 g, 0.0035 moles), EGDMA (3.36 g, 0.0169 moles) and BOC-phenylalanine (0.15 g, 0.00056 moles) were mixed with AIBN (0.5 moles). After purging for 30 minutes the monomer mixture was placed under UV light for 16 hours to yield a transparent block of polymer.

F) 2,2′ bi-pyridyl MIP 2,2′ bi-pyridyl is used in a number of applications such as DNA binding anti-cancer drugs (Laserna, J. J. ‘Modern Techniques in Raman Spectroscopy’ J. Wiley and Sons (1996), Ferraro, J. R. and Nakamoto, K. ‘Introductory Raman Spectroscopy’ Academic Press (1994), Al-Obaidi, A. H. R., Coates, C. G., Jacquet, L., McGarvey, J. J., Bell, S. E. J. and Kelly, J. M. (1997) ‘Resonance Raman Probing of the Interaction between dipyridophenazine complexes of Ru(II) and DNA’ J. Am. Chem. Soc).

### Example 2

### Raman Spectra of MIP's

The following experiments describe experiments investigating the effect of various factors upon Raman spectra of copolymers of EGDMA and MAA imprinted with a range of different templates. In all cases the effect of ethanol added to the copolymer/template complex and varying the amount of template present was investigated.

A) Raman Spectra of MIP Imprinted with BOC-phenylalanine

MIP imprinted with BOC-phenylalanine was prepared according to Example 1E. To characterise the Raman spectra of the imprinting molecules, the Raman spectra was recorded in the presence of ethanol (FIG. 2a), with ethanol alone (FIG. 2b) and the subtracted spectra is demonstrated by the spectrum in FIG. 2c. From this Figure it can be seen that BOC-phenylalanine is characterised by the presence of a strong Raman band centred around 1000 cm⁻¹.

The Raman spectra of the MIP imprinted with BOC-phenylalanine at various template:polymer ratios (4.55%, 0.93% and 0.46%) is shown in FIG. 3, including the spectra resulting from non-imprinted polymer (FIG. 3a) and BOC-phenylalanine alone (FIG. 3e). It can be seen from this Figure that the intensity of the band previously shown to be attributable to BOC-phenylalanine from the results of FIG. 2 (1000 cm⁻¹) is enhanced with increasing concentrations of template within the MIP's.

B) Raman Spectra of MIP Imprinted with Triazine

MIP imprinted with triazine was prepared according to Example 1D. To characterise the Raman spectra of the imprinting molecules, the Raman spectra was recorded in the presence of ethanol (FIG. 4a), with ethanol alone (FIG. 4b) and the subtracted spectra is demonstrated by the spectrum in FIG. 4c. From this Figure it can be seen that triazine is characterised by the presence of a strong Raman band centred around 1128 cm⁻¹.

The Raman spectra of the MIP imprinted with triazine at various template:polymer ratios (6.25%, 3.16% and 1.54%) is shown in FIG. 5, including the spectra resulting from non-imprinted polymer (FIG. 5a) and triazine alone (FIG. 5e). It can be seen from this Figure that the intensity of the band previously shown to be attributable to triazine from the results of FIG. 4 (1128 cm⁻¹) is enhanced with increasing concentrations of template within the MIP's.

C) Raman Spectra of MIP Imprinted with 2,2′ bi-pyridyl MIP imprinted with 2,2′ bi-pyridyl was prepared according to Example 1F. To characterise the Raman spectra of the imprinting molecules, the Raman spectra was recorded in the presence of ethanol (FIG. 6a), with ethanol alone (FIG. 6b) and the subtracted spectra is demonstrated by the spectrum in FIG. 6c. From this Figure it can be seen that 2,2′ bi-pyridyl is characterised by the presence of a number of strong Raman bands.

The Raman spectra of the MIP imprinted with 2,2′ bi-pyridyl at various template:polymer ratios (6.25%, 3.03% and 1.54%) is shown in FIG. 7, including the spectra resulting from non-imprinted polymer (FIG. 7a) and 2,2′ bi-pyridyl alone (FIG. 7e). It can be seen from this Figure that the intensity of the bands previously shown to be attributable to 2,2′ bi-pyridyl from the results of FIG. 6 are enhanced with increasing concentrations of template within the MIP's.

### Example 3

### Raman Spectroscopy for Monitoring the Presence and Removal of Templates in MIP's

MIP's imprinted with BOC-phenylalanine, triazine and 2,2′ bi-pyridyl were washed in ethanol using a mechanical shaker and the removal of these templates was then monitored using Raman spectroscopy. After said removal, the polymers were then rebound with the original template in ethanol solution at a high concentration (10% w/v) and the rebinding effect was again monitored using Raman spectroscopy. As a control, the above procedure was similarly carried out for a non-imprinted polymer (prepared according to Example 1C).

A) Monitoring the Presence and Removal of BOC-phenylalanine

MIP imprinted with BOC-phenylalanine and non-imprinted polymer were prepared according to Examples 1E and 1C, respectively.

Spectra were then recorded for non-imprinted polymer (FIG. 8a), after washing with ethanol (FIG. 8b) and then when rebound with template (FIG. 8c). No significant changes were observed in the region of 1000 cm⁻¹ (characteristic of BOC-phenylalanine as shown in FIG. 2) which indicated that either no binding of the template had occurred or that the concentration of template was not significant enough in concentration to appear in the Raman spectra of the bulk polymer. FIG. 8c also shows some down shifting in bands caused by cracks observed on the polymers.

A similar procedure was carried out on the MIP imprinted with BOC-phenylalanine. It was observed that the band present at 1000 cm⁻¹ decayed during washing in ethanol (FIG. 8e) when compared to the spectra prior to washing (FIG. 8d), however, the band was seen to recover almost back to the original level during re-binding (FIG. 8f). Therefore, re-binding occurred only for MIP's imprinted with the BOC-phenylalanine.

The data obtained from FIG. 8 was extracted and the relative intensity of the Raman band associated with the template at ˜1000 cm⁻¹ was plotted against the removal and re-binding time and can be seen in FIG. 9. It was observed that 92% of the template was removed during washing for 60 hours (a long duration due to the requirement of the solvent to penetrate large distances of heavily cross linked polymer) and rebinding between BOC-phenylalanine and MIP similarly occurred after 60 hours.

B) Monitoring the Presence and Removal of Triazine

MIP imprinted with triazine and non-imprinted polymer were prepared according to Examples 1D and 1C, respectively. This experiment was carried out in an identical fashion to that of Example 3, step A) above and the removal/re-binding spectra for non-imprinted polymer and MIP imprinted with triazine can be seen in FIGS. 10a-c and 10d-f, respectively. As with the BOC-phenylalanine experiment, re-binding occurs only with MIP imprinted with triazine and there was no significant binding between the non-imprinted polymer and the triazine.

The data obtained from FIG. 10 was then extracted and the relative intensity of the Raman band associated with the template at ˜1128 cm⁻¹ was plotted against the removal and re-binding time and can be seen in FIG. 11. It was observed that 60% of the template was removed during washing for 60 hours, which was much less than was removed in the BOC-phenylalanine experiment reflecting the high molecular recognition properties of these imprinted polymers.

C) Monitoring the Presence and Removal of 2,2′ bi-pyridyl Template

MIP imprinted with 2,2′ bi-pyridyl and non-imprinted polymer were prepared according to Examples 1F and 1C, respectively. This experiment was carried out in a similar fashion to that of Example 3, steps A) and B) above and the removal/re-binding spectra for non-imprinted polymer and MIP imprinted with 2,2′ bi-pyridyl can be seen in FIGS. 12 and 13, respectively. As with the previous experiments, re-binding occurs only with MIP imprinted with 2,2′ bi-pyridyl and there was no significant binding between the non-imprinted polymer and the 2,2′ bi-pyridyl.

The data obtained from FIGS. 12 and 13 was then extracted and the relative intensity of the Raman band associated with the template at 1573 cm⁻¹ was plotted against the removal and re-binding time and can be seen in FIG. 14. It can be observed from this Figure that removal/rebinding of the 2,2′ bi-pyridyl is almost fully recoverable.

### Example 4

### Raman Spectroscopy of Powdered MIP's

The results from Example 3 demonstrate that the use of block polymer MlP's is useful to detect conformational changes and confirm the effectiveness of this detection technique. However, the binding and washing steps require very long incubation times (typically 60 hours ) due to the time taken for diffusion through the entire block.

A) Washing and Rebinding of Powdered MIP's Imprinted with 2,2′ bi-pyridyl

A washing and rebinding investigation was performed using powdered MIP's, focusing especially on MIP's imprinted with 2,2′ bi-pyridyl which were prepared according to the following protocol:

2,2′ bi-pyridyl was dissolved in a monomer mixture containing MAA (1.744 mmoles), EGDMA (8.48 mmoles) and AIBN (90 Emoles) and polymerized under UV light at 366 nm). Polymers containing 0.025 g (0.16 mmoles), 0.05 g (0.32 mmoles) and 0.1 g (0.64 mmoles) of 2,2′ bi-pyridyl were prepared. The monomer:template ratios in these polymers were 64:1, 32:1 and 16:1 respectively.

For this investigation, polymers with a 16:1 ratio of monomer:template were ground up with a pestle and mortar and sieved to give particle size fractions of 212-150, 150-106, 106-45 and <45 μm. A washing and rebinding investigation was then performed using 2,2′ bi-pyridyl imprinted polymers with a particle size of 185 μm.

FIGS. 15 and 16 indicate that a reduction in the intensity of the Raman bands corresponding to 2,2′ bi-pyridyl (1573 and 1591 wavenumber/cm⁻¹) is observed with increased washing time. It appears from these Figures that only 3 minutes is required to remove or bind 75% of the 2,2′ bi-pyridyl template in powder form, compared with 120 hours for the block polymer imprinted with 2,2′ bi-pyridyl (see FIGS. 13 and 14). Therefore, the use of powdered MIP's decreases the removal/binding time by a factor of approximately 2400 times.

These results indicate that complete removal of 2,2′ bi-pyridyl from the powdered MIP takes approximately 5 minutes.

B) Washing and Rebinding of Non-imprinted Powdered MlP's

Non-imprinted polymer was prepared according to Example 1C and then powdered in an identical manner to the 2,2′ bi-pyridyl imprinted MlP's in Example 4A.

FIG. 17 shows the results of the washing and rebinding investigation using this non-imprinted polymer wherein a visible amount of 2,2′ bi-pyridyl has been incorporated into the polymer after a 10 minute incubation period, which is then totally removed after only 1 minute of washing. This indicates that 2,2′ bi-pyridyl is predominantly associated with surface binding and has a much weaker molecular interaction and confirming that it is easier to remove or wash a template from a non-imprinted polymer than from an imprinted polymer.

C) Particle Size Investigation

Powdered MIP's imprinted with 2,2′ bi-pyridyl were prepared according to Example 4A. The influence of the particle size of these powdered MIP's on washing was studied for the 185±35 μm, 130±15 μm, 90±15 μm, 60±15 μm and <45 μm particle size range of MIP's.

FIG. 18 shows the differing relative Raman intensities of the 2,2′ bi-pyridyl peak obtained for varying washing times with the different particle sizes of powdered MIP's. Generally, this Figure demonstrates that the smallest particle sizes of MIP's required a shorter duration of washing to remove the 2,2′ bi-pyridyl template from the powdered polymer.

Graphs were then constructed to demonstrate the kinetics of binding by equating the MIP particle size (FIG. 19A) and the surface area (FIG. 19B) with the template removal time. These graphs indicate that the removal of 2,2′ bi-pyridyl template is dependent upon the particle size of the MIP, wherein templates are washed from polymers with smaller particle sizes more rapidly.

### BRIEF DESCRIPTION OF THE DRAWINGS

FIG. 1: Schematic diagram of Raman experimental system for a tuneable system.

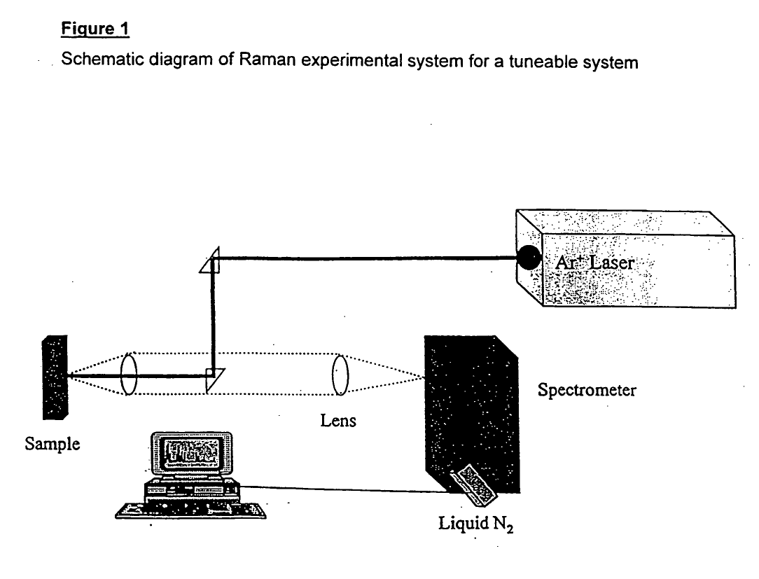

FIG. 2: Raman spectra for a BOC-phenylalanine solution

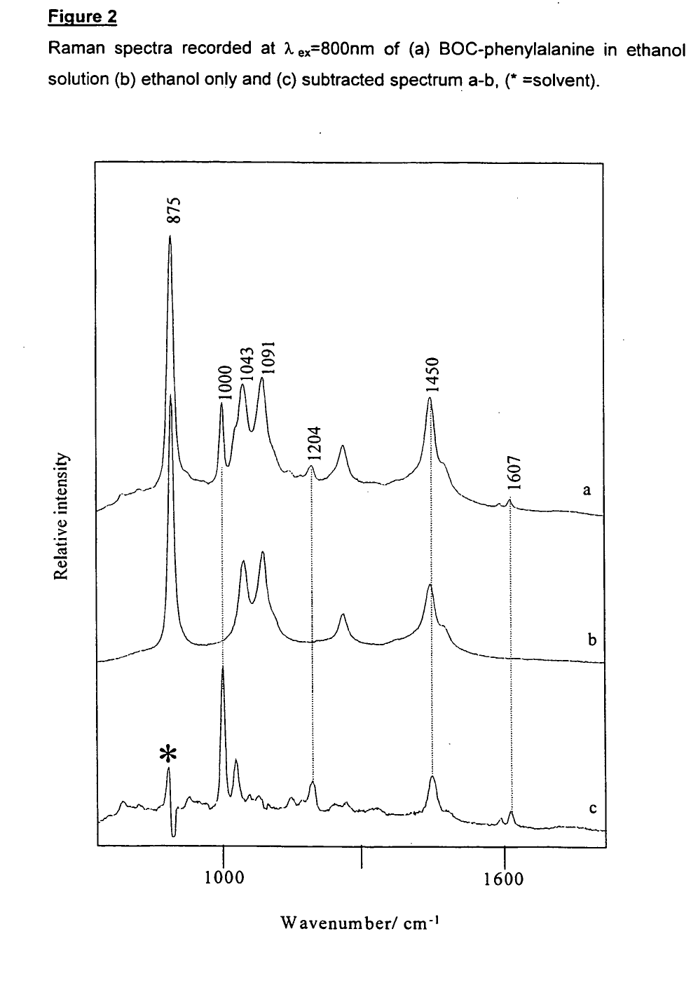

FIG. 3: Raman spectra for MIP imprinted with BOC-phenylalanine

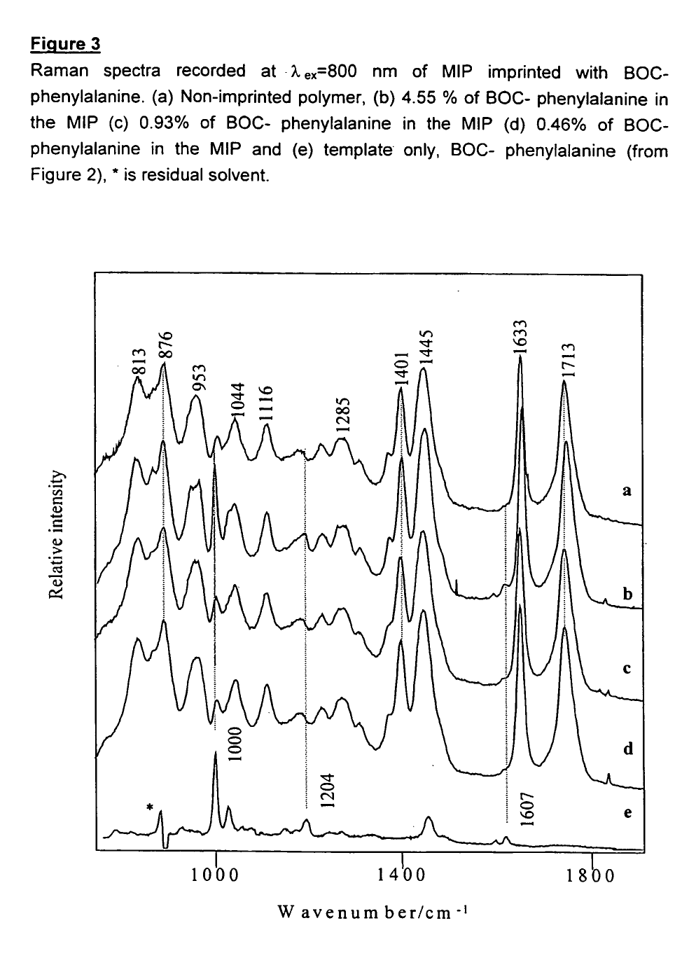

FIG. 4: Raman spectra for a triazine solution

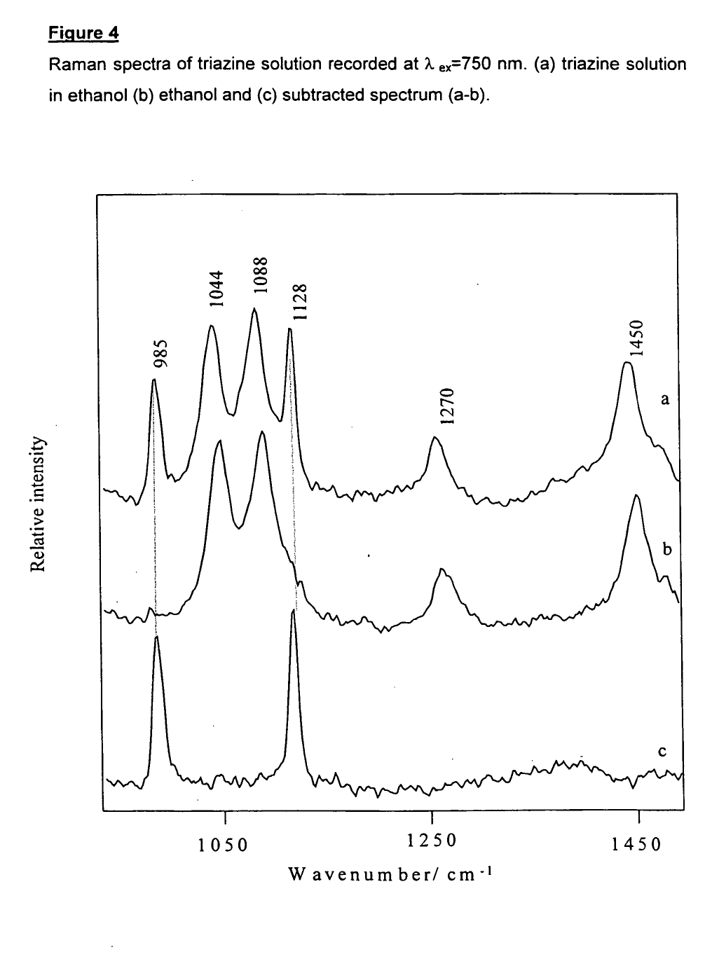

FIG. 5: Raman spectra for MIP imprinted with triazine

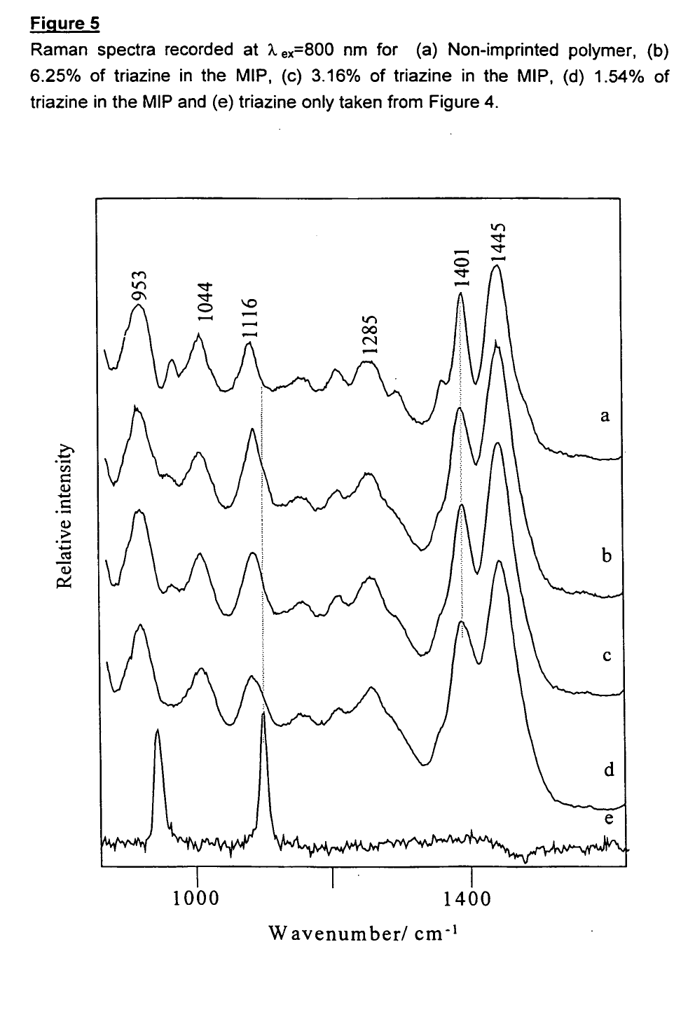

FIG. 6: Raman spectra for a 2,2′ bi-pyridyl solution

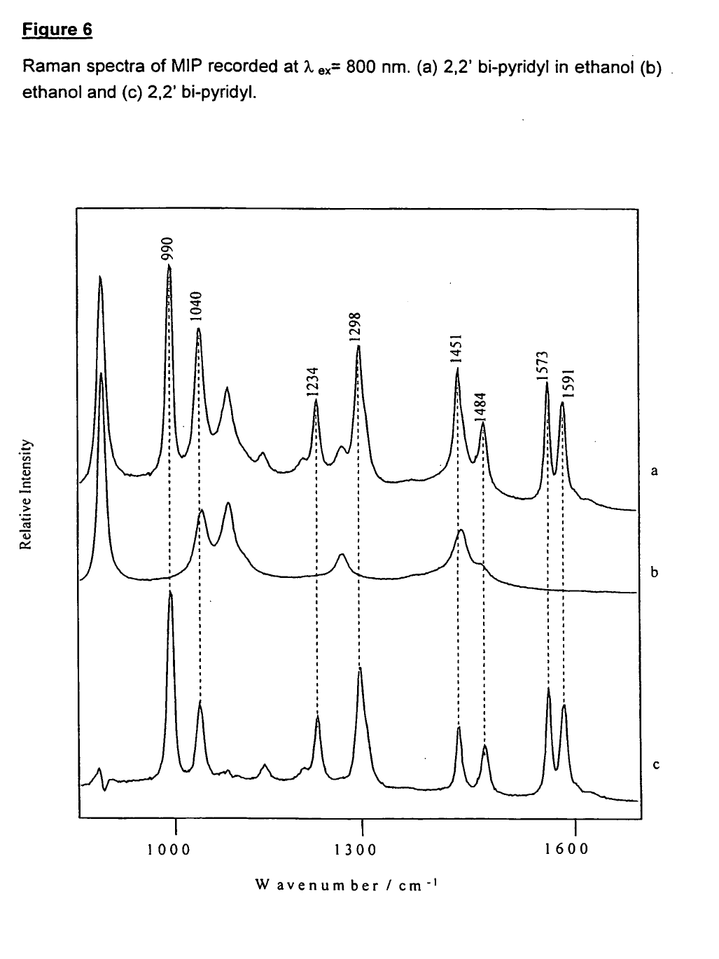

FIG. 7: Raman spectra for MIP imprinted with 2,2′ bi-pyridyl

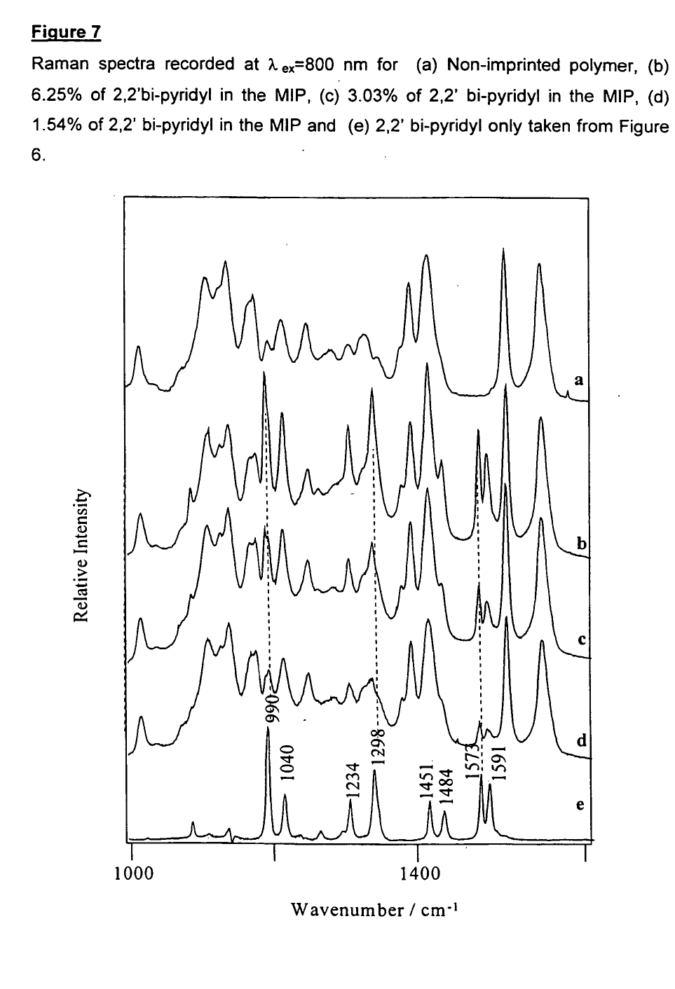

FIG. 8: Raman spectra demonstrating the effect of binding, washing and rebinding of BOC-phenylalanine on non-imprinted and imprinted MlP's.

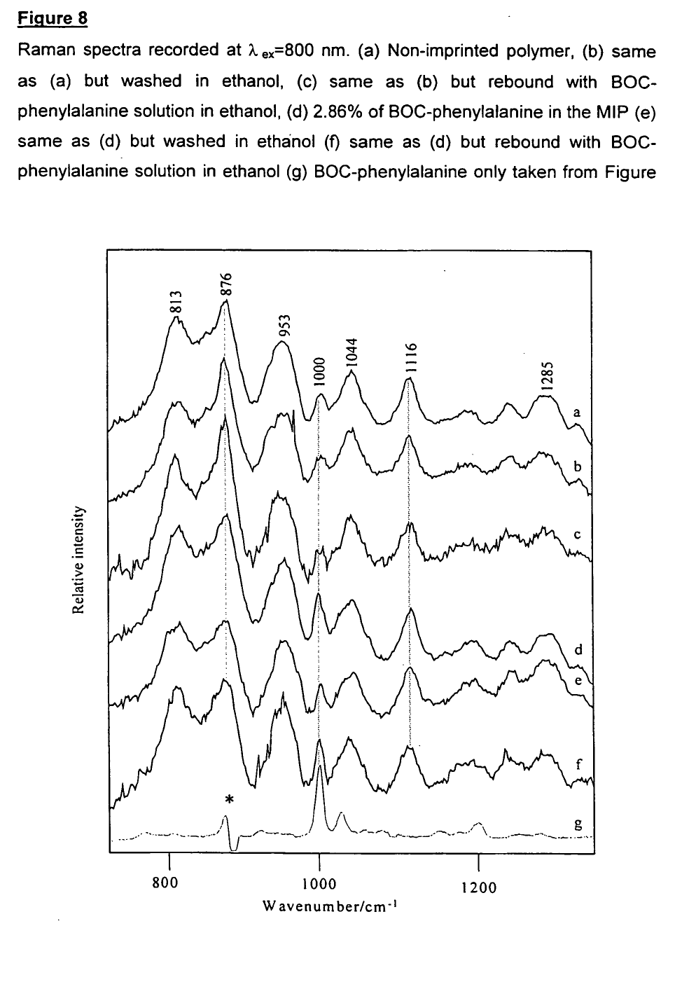

FIG. 9: Graph demonstrating the intensity change upon washing and rebinding of MIP imprinted with BOC-phenylalanine and non-imprinted polymer.

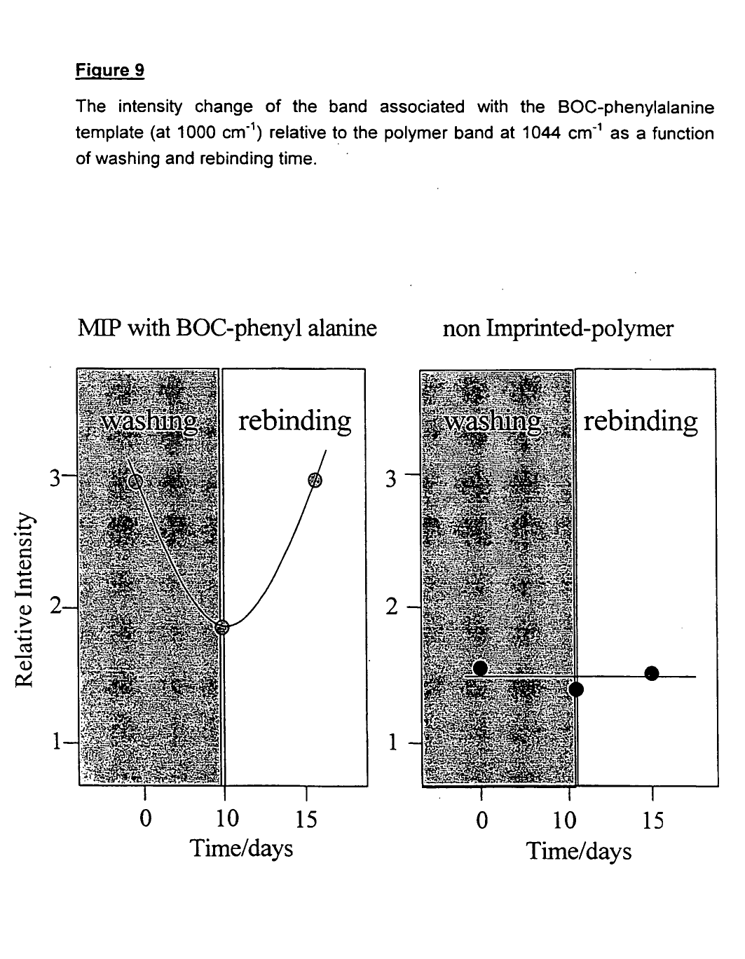

FIG. 10: Raman spectra demonstrating the effect of binding, washing and rebinding of triazine on non-imprinted and imprinted MlP's.

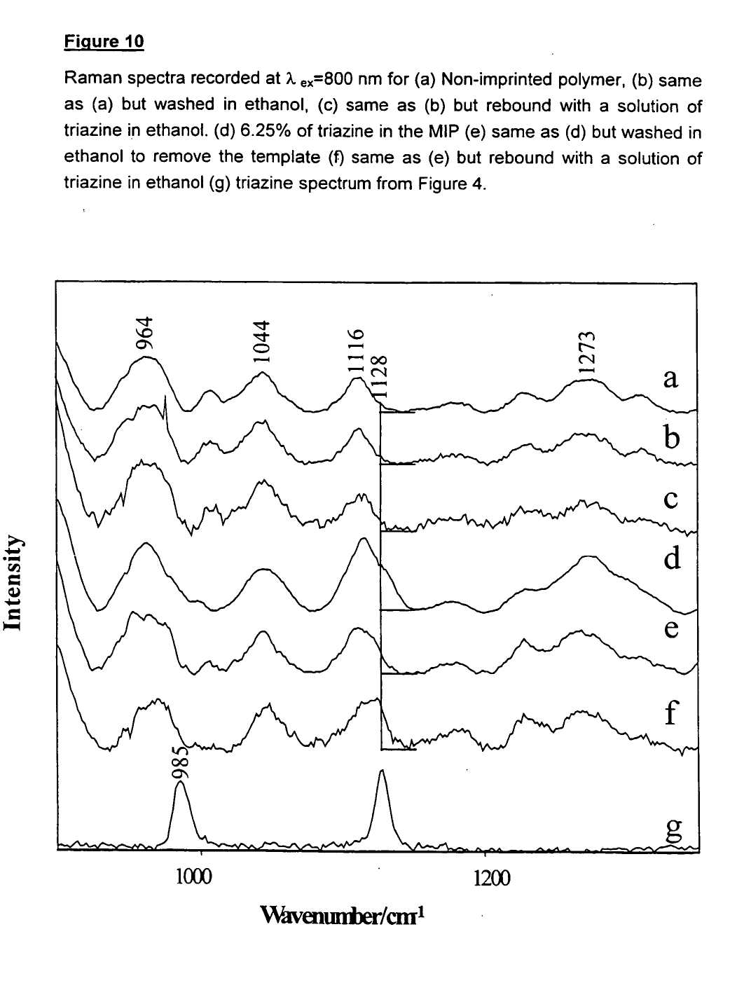

FIG. 11: Graph demonstrating the intensity change upon washing and rebinding of MIP imprinted with triazine and non-imprinted polymer.

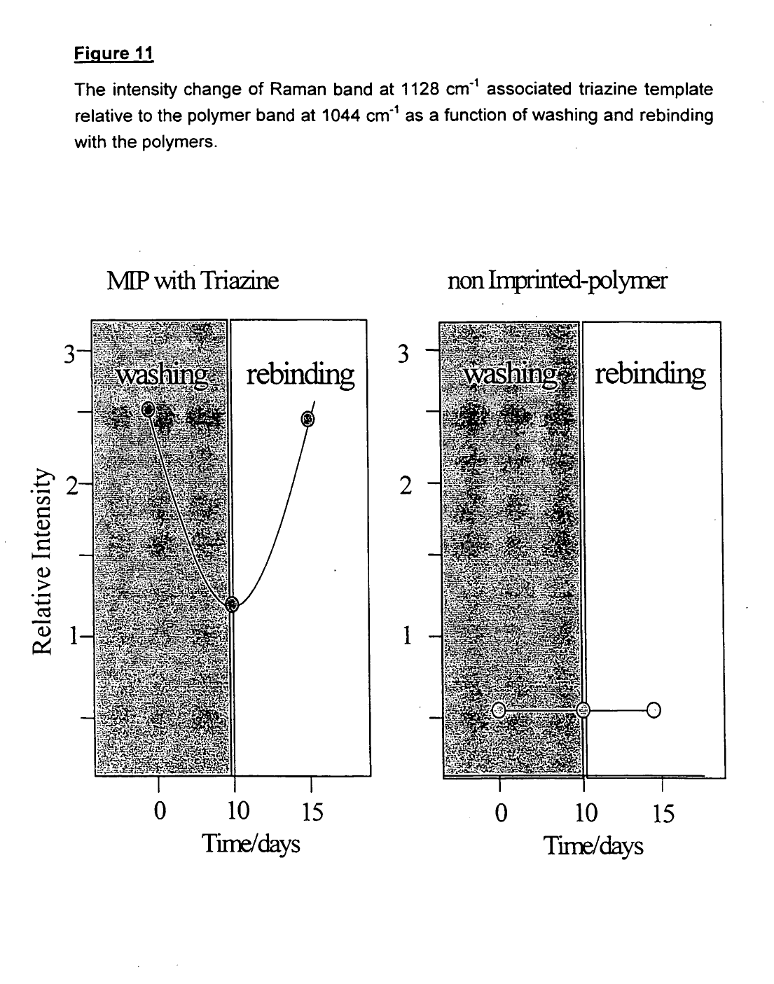

FIG. 12: Raman spectra demonstrating the effect of binding, washing and rebinding of 2,2′ bi-pyridyl on non-imprinted MlP's.

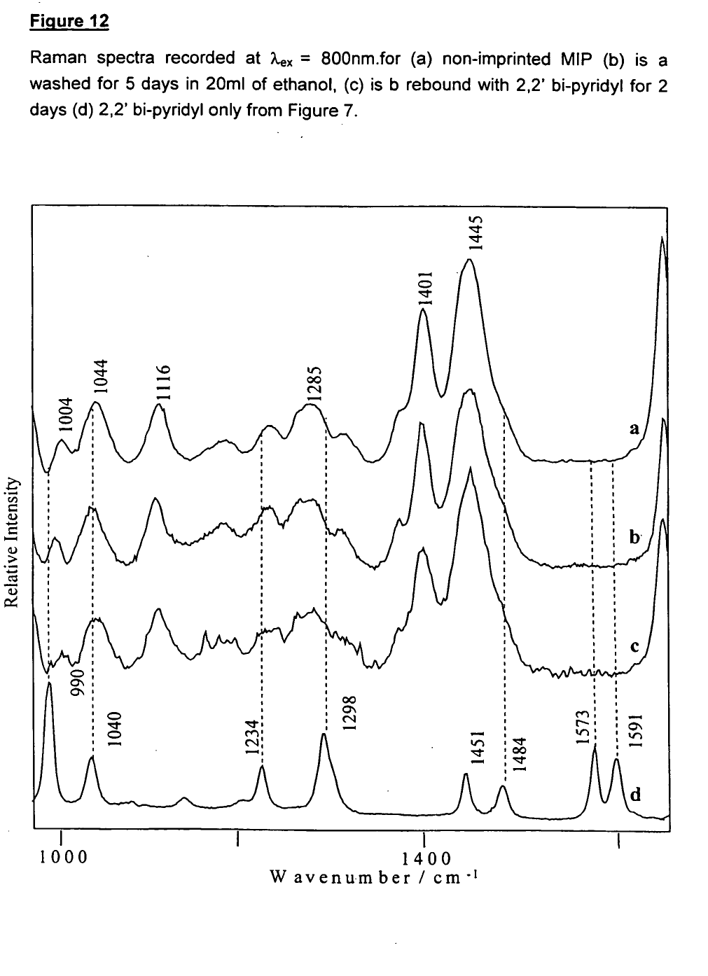

FIG. 13: Raman spectra demonstrating the effect of binding, washing and rebinding of 2,2′ bi-pyridyl on imprinted MIP's.

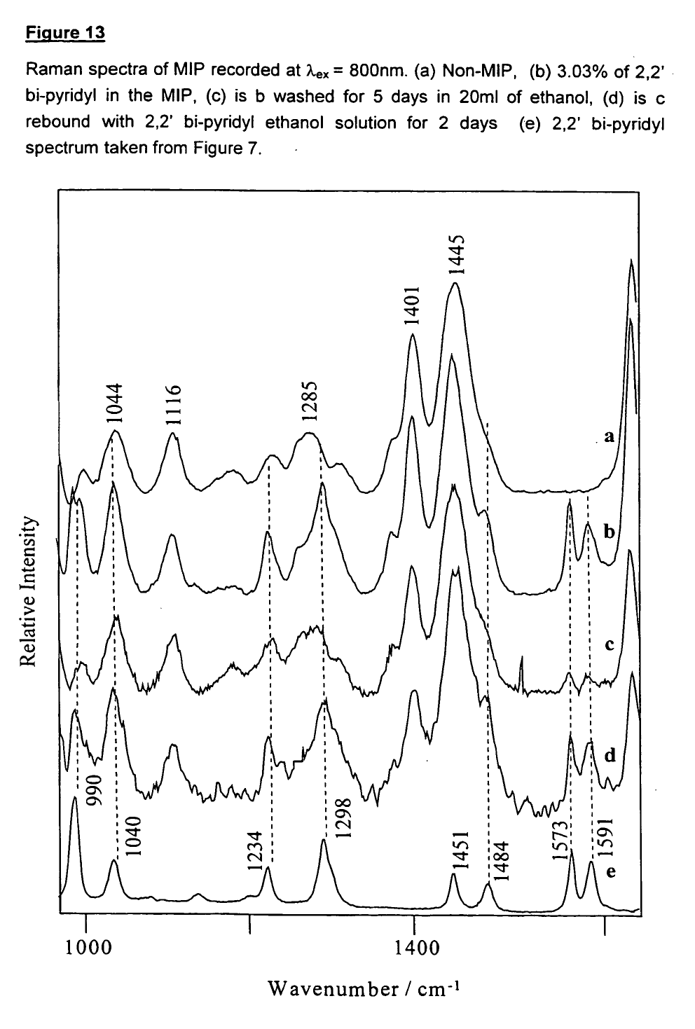

FIG. 14: Graph demonstrating the intensity change upon washing and rebinding of MIP imprinted with 2,2′ bi-pyridyl and non-imprinted polymer.

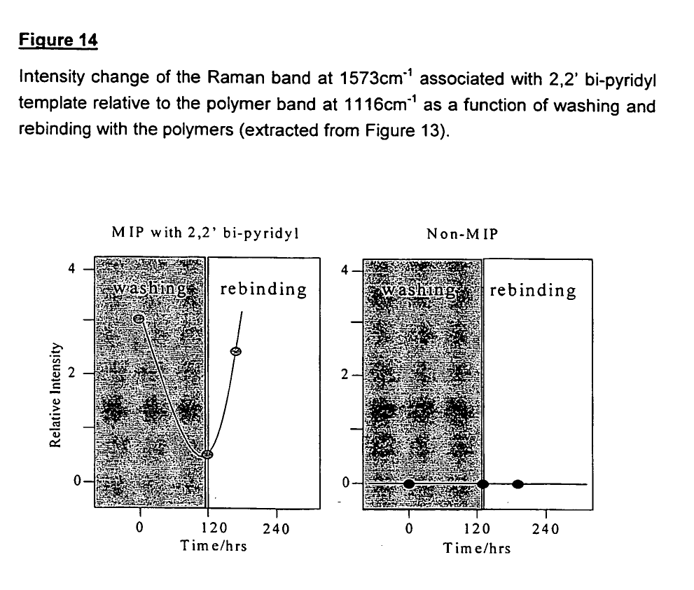

FIG. 15: Raman spectra demonstrating the intensity change upon washing of powdered MIP's imprinted with 2,2′ bi-pyridyl.

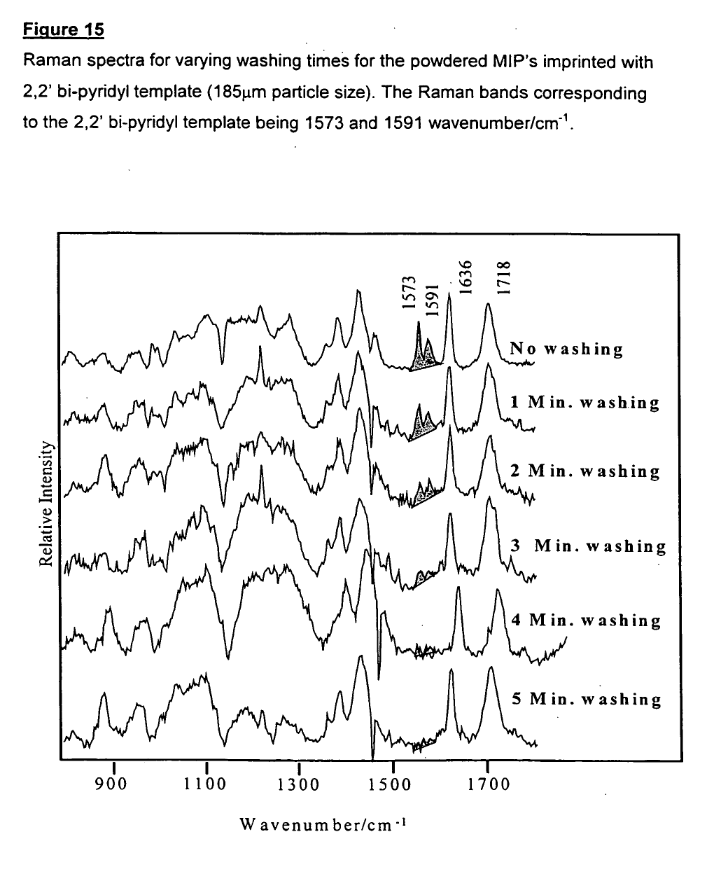

FIG. 16: Raman spectra demonstrating the intensity change upon rebinding of powdered MIP's imprinted with 2,2′ bi-pyridyl.

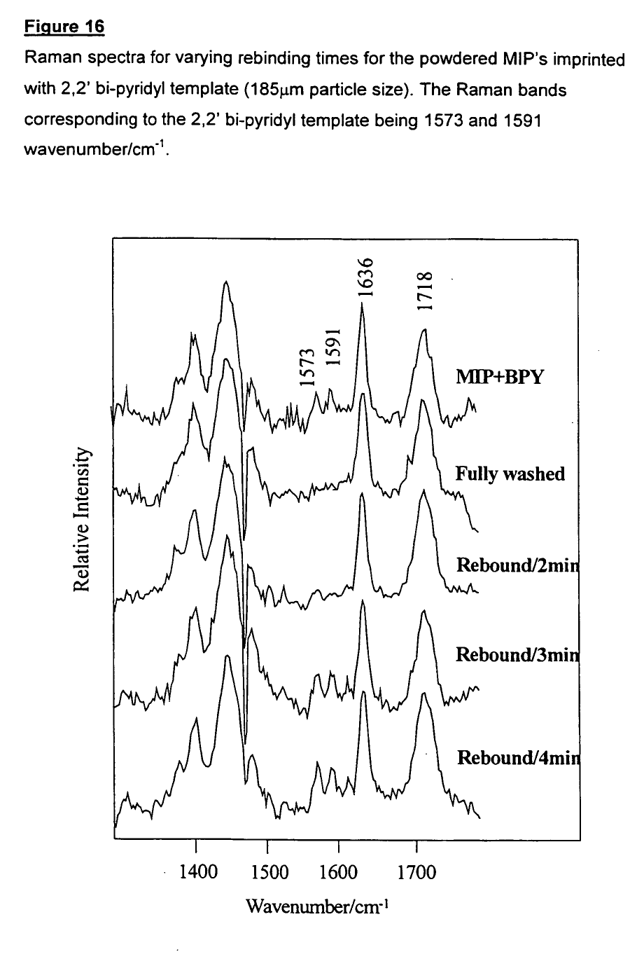

FIG. 17: Raman spectra demonstrating the effect of binding and washing on a non-imprinted powdered polymer.

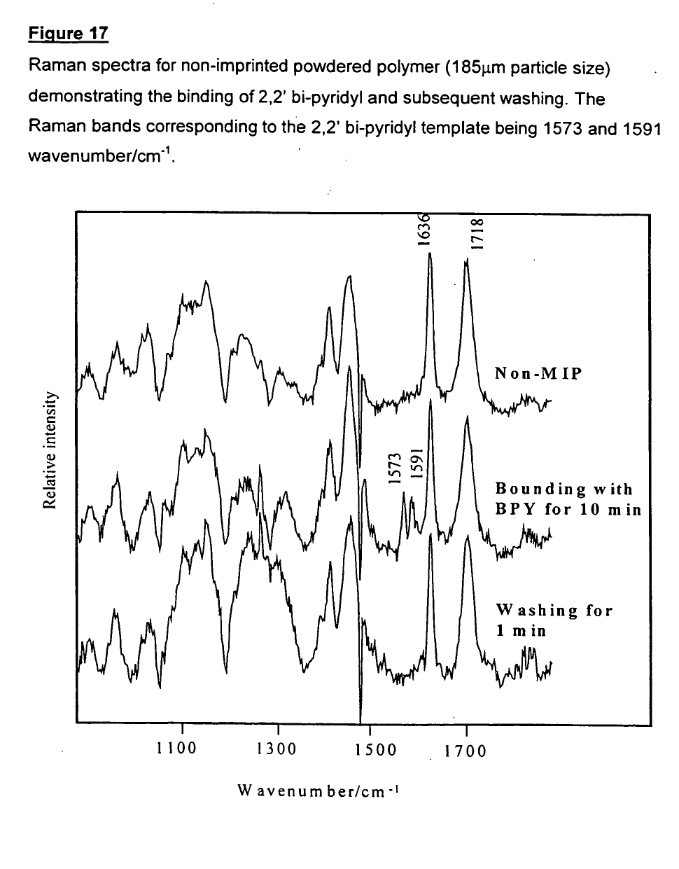

FIG. 18: Differing relative Raman intensities of the 2,2′ bi-pyridyl peak obtained for varying washing times with differing particle sizes of powdered MIP's.

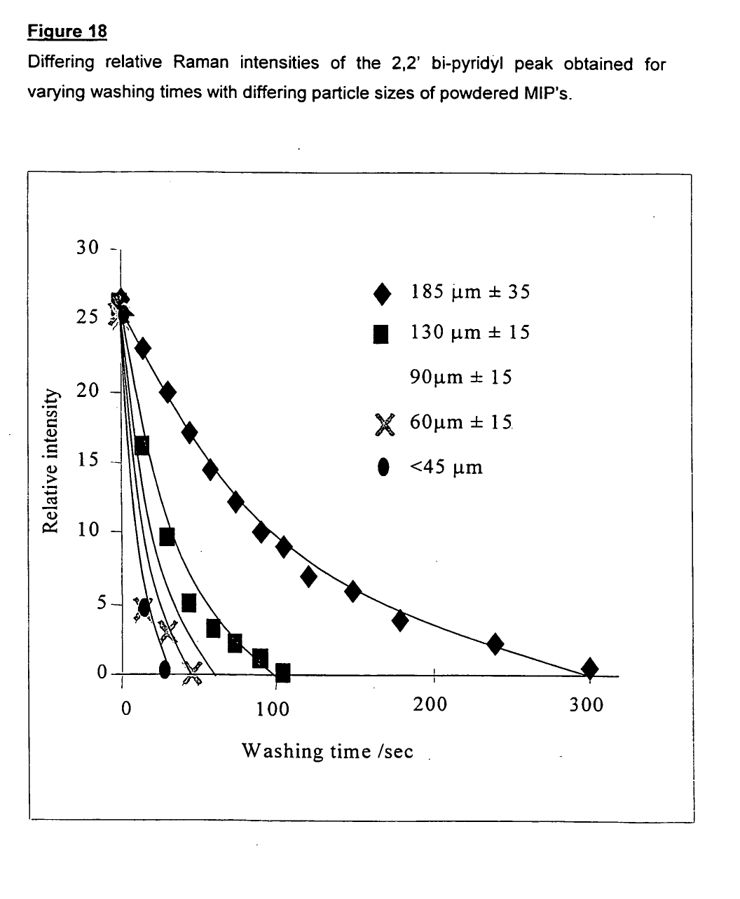

FIG. 19: Graphs demonstrating the effect of MIP particle size (A) and surface area (B) upon template removal time.

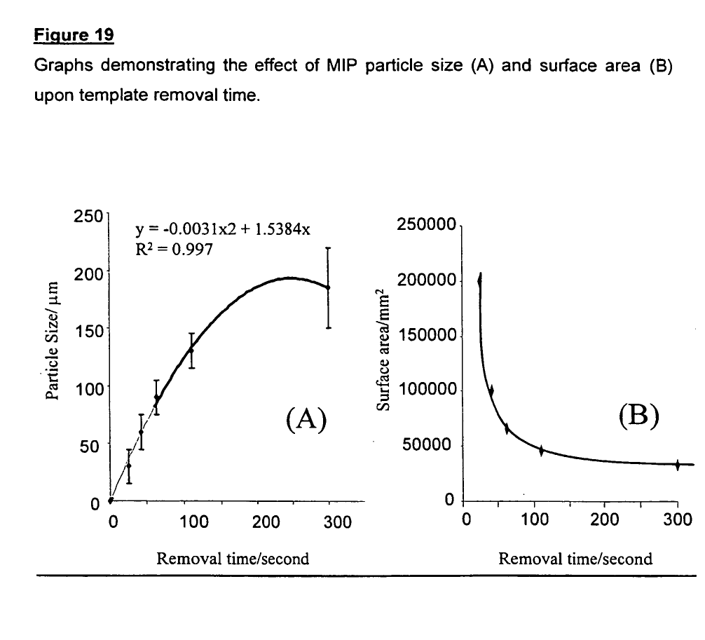

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

1. A process for detecting interactions involving a biological molecule which comprises the preparation of a polymer molecularly imprinted with a biological molecule and the use of Raman spectroscopy applied to said polymer as an optoelectronic technique for sensing said interactions.

2. A process according to claim 1 wherein the biological molecule is selected from antibodies, antigens, polynucleotides, enzymes, enzyme substrates and analogues, receptors and ligands and drugs or other pharmaceutically useful substances.

3. A process according to claim 1 for detecting interactions between antibodies and antigens which comprises the preparation of a polymer molecularly imprinted with an antigen to form an artificial antibody and the use of Raman spectroscopy applied to said polymer as an optoelectronic technique for sensing interactions between antigens and the artificial antibody.

4. A process according to claim 1 for detecting interactions between enzymes and enzyme substrates which comprises the preparation of a polymer molecularly imprinted with a substrate to form an artificial enzyme and the use of Raman spectroscopy applied to said polymer as an optoelectronic technique for sensing interactions between substrates and the artificial enzyme.

5. A process according to claim 1 for detecting interactions between receptors and ligands which comprises the preparation of a polymer molecularly imprinted with a ligand to form an artificial receptor and the use of Raman spectroscopy applied to said polymer as an optoelectronic technique for sensing the interactions between ligands and the artificial receptor.

6. A process according to any one of claims 1 to 5 wherein the molecularly imprinted polymer (MIP) is prepared covalently or non-covalently.

7. A process according to claim 6 wherein the MIP is prepared non-covalently.

8. A process according to any one of claims 1 to 7 wherein the MIP is prepared non-covalently in the presence of one or more functional polymers and a biological molecule.

9. A process according to claim 8 wherein the functional monomers include methacrylic acid, ethylene glycol dimethacrylate or azoisobutyronitrile.

10. A process according to claim 8 or claim 9 wherein the MIP is prepared by polymerisation of functional monomers which polymerisation occurs at suitable conditions eg. low or high temperatures and in the absence of solvent.

11. A process according to claim 10 wherein polymerisation occurs using UV irradiation or electro-polymerisation.

12. A process according to any one of claims 1 to 11 wherein the biological molecule is cleaved from the MIP, or otherwise removed, by the addition of a lower alkyl alcohol.

13. A process according to claim 12 wherein cleavage is assisted by mechanical agitation, ultrasound, shock waves, or any other suitable agitation means.

14. A process according to any one of claims 1 to 13 wherein the polymer is present as bulk polymer, a thin film, be ground up as particles or polymerised directly into spherical particles.

15. A process for detecting interactions involving a biological molecule which comprises:
(a) preparing a MIP capable of functioning as a complement to a biological molecule by incubation of functional monomers with said biological molecule;
(b) removal of the biological molecule from the MIP;
(c) comparing the Raman spectra of the MIP with the Raman spectra of a non-imprinted polymer to determine the wavelength of the peak associated with the biological molecule (“relevant peak”);
(d) introducing the MIP into an assay mixture which contains the biological molecule, incubating it therein and then obtaining a Raman spectrum of the MIP; and
(e) comparing the intensity of the relevant peak on the Raman spectra obtained from incubation of the MIP with the biological molecule in step (d) with the relevant peak obtained from the Raman spectra of the MIP alone in step (b);
wherein any change in intensity of the relevant peak will indicate existence of an interaction between the biological molecule and the complement of the biological molecule.

16. A process according to claim 15 for performing enzyme binding assays wherein the biological molecule and the complement of the biological molecule are enzymes and substrates.

17. A process according to claim 15 for performing enzyme binding assays wherein the biological molecule and the complement of the biological molecule are enzymes and substrate analogues.

18. A process according to claim 15 for monitoring conformational changes wherein the biological molecule and the complement of the biological molecule are receptors and ligands.

19. A process according to claim 15 for performing immunoassays wherein the biological molecule and the complement of the biological molecule are antibodies and antigens.

20. A process for quantifying the amount of a biological molecule present in a given environment which comprises:
(a) preparing a MIP capable of functioning as a complement to a biological molecule by incubation of functional monomers with said biological molecule;
(b) removal of the biological molecule from the MIP;
(c) comparing the Raman spectra of the MIP with the Raman spectra of a non-imprinted polymer to determine the wavelength of the peak associated with the biological molecule (“relevant peak”);
(d) measuring the intensity of the relevant peak on the Raman spectra of a range of concentrations of the polymer molecularly imprinted with the biological molecule;
(e) plotting a graph of relative peak intensity of the relevant peak against concentration of the biological molecule;
(f) introducing the MIP into an assay mixture which contains an unknown concentration of the biological molecule, incubating it therein and then obtaining a Raman spectrum of the MIP;
(g) calculating the concentration of the biological molecule by reference of the relative intensity of the relevant peak in (f) to the graph constructed in (e).

21. A process according to claim 20 for quantifying the amount of a biological molecule present in a given environment wherein said biological molecule is a drug or other pharmaceutically useful substance.
